(19) United States
(12) Patent Application Publication
(10) Pub. No.: US 2008/0033454 A1
(43) Pub. Date: Feb. 7, 2008

(54) Device for Holding and /or Guiding Instruments or Cannulas

(76) Inventors: Andreas Lukoschek, Bottrop (DE); Thomas Remmele, Ubstadt Weiher (DE); Siegfried Horn, Karlsruhe (DE); Bernd Gutmann, Karlsruhe (DE)

Correspondence Address: WILLIAM COLLARD;COLLARD & ROE, P.C., 1077 NORTHERN BOULEVARD, ROSLYN, NY, 11576, US

(21) Appl. No.: 11/795,939

(22) PCT Filed: Jan. 24, 2006
(86) PCT No.: PCT/DE06/00104

§ 371 Date: Jul. 24, 2007

(30) Foreign Application Priority Data
Jan. 24, 2005 (DE) 10 2005 003 380.6

(51) Int. Cl. A61B 17/00

(52) U.S. Cl. 606/130; 600/417

(57) ABSTRACT

The invention relates to a holder device (1) for medical instruments, including endoscopes, or cannulas, such as those that are used within the framework of minimally invasive surgery or interventional radiology. Proceeding from a state of the art in which the treating physician knows holder devices (1) and guidance systems, an improved holder device (1) is proposed within the framework of the invention, which makes it possible, at any time, to uncouple the holder device (1) and, if applicable, the guidance system connected with the holder device (1), from the medical instruments or cannulas held with the holder device (1), without having to retract or remove the instruments or cannulas.

Device for holding and/or guiding medical instruments, including endoscopes, or cannulas, which are introduced into a patient's body with a defined orientation, by way of interpunction, preferably in the sector of interventional radiology or minimally invasive surgery, and subsequently driven forward up to a defined penetration depth, within the patient's body, if necessary.

Such a holder device, a so-called “needle trocar,” is previously known from DE 198 08 220 A1, among other things. As is accurately stated in the introduction to the specification of this document, it is necessary in modern radiology, for example for the purpose of conducting biopsies, to carry out corresponding functions of the patient's body, whereby the needles used for this purpose must be precisely positioned. For this purpose, a stereotactic needle holder device is previously known from the aforementioned patent application, which is supposed to be used to place such a needle in as defined a manner as possible, whereby if necessary, an imaging system, such as a nuclear resonance tomograph, can be used. It is perceived as being problematical, within the scope of the previously known application, that the imaging systems usually do not fix their reference points on the patient's body, but rather on the operating table. In the case of a relative movement of the patient with regard to the operating table, the needle positioning is therefore necessarily defective. This is not acceptable under any circumstances in the medical sector. The solution proposed in this regard therefore comprises a clamping device having an adjustable channel, whereby the clamping device as a whole can be glued onto the patient's skin. The needle holder itself comprises a ball joint connected with an attachment plate, which mounts an instrument introduction tube in articulated manner. The instrument introduction tube can then be fixed in place in the as fixed positioning by means of a clamping device assigned to the ball joint.

A similar solution is provided by DE 297 19 526 U1. Essentially, the neuro-trocar device disclosed there differs from the needle trocar device already described in that the attachment plate already mentioned can be attached not to the patient's body, but rather to the patient's head.

Another aid for positioning and finally, also introduction of medical devices into the patient's body, which is certainly valuable, is previously known from DE 196 17 534 A1. The patent application describes a positioning grid that has cavities for accommodating contrast agents. The contrast agents accommodated in the cavities of the positioning grid in turn ensure that the positioning grid, i.e. its cavities can be imaged in an imaging system, in other words by means of a nuclear magnetic resonance tomograph, for example. The positioning grid, which is easily visible in the imaging system in this manner, can therefore be used to select and predetermine a medically logical punction point on the patient's body, using the imaging system, in that the positioning grid is glued onto the skin of the patient's body at the selected location.

Fundamentally, it is perceived as being problematical in connection with the positioning aids already mentioned that a possible incorrect orientation or incorrect positioning can only be corrected with difficulty. The use of mechanical or remote-controlled guide systems generally fails because spontaneous intervention by the treating physician can only be corrected in extremely complicated manner, or not at all, because of the fixed connection between the guide system and the medical instrument or cannula. In most cases, it is necessary to completely remove the medical instrument or the cannula that has already been introduced into the patient's body, and to start the intervention right from the beginning again, i.e. with a new interpunction. Another complication fundamentally consists in the fact that of course, patient movements cannot and should not be precluded with certainty. Furthermore, in the case of interventions involving respiratory organs, a certain relative movement of the patient with regard to the instrument is to be accepted. Fundamentally, it is a requirement for modern systems of radiology, as well as for minimally invasive surgery, that any guidance and positioning system that supports the treating physician should not impair or hinder natural patient movements, or actually make them impossible.

The invention is therefore based on the task of creating a holder device for medical instruments or cannulas that permits intervention by the treating physician at all times, in other words even after introduction of the instrument or the cannula into the patient's body, and does not further impair possible patient movements, and, at the same time, guarantee optimal support of the treating physician.

The solution of the task according to the invention succeeds in that the holder device according to the invention, in contrast to the holder devices known from the state of the art, can be released from the instrument and/or the cannula at all times, but particularly also after interpunction has taken place, in accordance with its intended purpose.

In this connection, the holder device according to the invention is connected with a mechanical guidance system, preferably in releasable manner, within the scope of the invention. This guidance system ideally works together with an imaging system, in other words possibly an MRI (magnetic resonance imaging) system or an MRT (magnetic resonance tomograph) system or a CT (computer tomography) system.

The above system is usually supplemented with display and operating elements, in other words possibly a touch screen or a computer having a graphic user interface, whereby the treating physician can establish the interpunction and intervention site using the imaging system and the aforementioned display and operating elements. In this way, positioning and orientation of the medical instrument or the cannula to be inserted is clearly provided.

The system can subsequently automatically calculate at what location and in what orientation of the patient's body the instrument or the cannula, respectively, must be set down. Subsequently, the connected guidance system can automatically move to the interpunction site of the patient's body. After the interpunction has taken place, preferably by the treating physician in the predetermined orientation, further introduction of the medical instrument or of the cannula at the interpunction site, in the predetermined orientation, is also carried out by the treating physician himself/herself. The guidance system according to the invention therefore intentionally and explicitly refrains from automating the punction and advance of the cannula or the medical instrument into the patient's body.

For the purpose of introduction of a cannula, the holder device used in this connection consists of a sleeve holder that essentially comprises an angular element that consists of a holding shoulder and a sleeve seat disposed at a right angle to it. The sleeve seat as such is provided with a passage for accommodating a segment of the sleeve, whereby here, the sleeve is already provided with a cannula that has been passed through. The passage of the sleeve seat for accommodating the corresponding sleeve segment or the sleeve as a whole is provided with a longitudinal slit over its entire length expanse. The sleeve holder can be released from the sleeve, and thereby the cannula can also be released from the sleeve holder, at any time, by way of this longitudinal slit, i.e. the proximal opening of the sleeve holder.

For this purpose, all that is necessary is for the sleeve to be moved in the proximal direction relative to the sleeve seat, until the sleeve comes out of engagement with the passage and subsequently, only the cannula is surrounded by the aforementioned passage of the sleeve seat. In this position, the cannula can easily be threaded out through the longitudinal slit of the passage, and thus a guidance system that might be connected can be completely uncoupled from the cannula. This is practical after an automatic interpunction has taken place, for example, if the cannula or the medical instrument has been inserted so far that it is held at the desired location and in the desired orientation more or less automatically by the skin and the patient's tissue.

Within the scope of the invention described here, special cannulas have been developed, which are already pre-packaged in such a manner that the cannula, complete with a sleeve and, if necessary, a sleeve holder, can be supplied as sterile disposable articles for one-time use. The advantage of a cannula already pre-assembled in a corresponding sleeve consists in the fact that otherwise, there is the risk that when the cannula is introduced into the sleeve, preferably made from plastic, a chip, possibly only a microscopic chip, might be removed by the cannula tip, and this could then be introduced into the patient's body with the cannula. Of course, any such introduction of a foreign body must absolutely be avoided within the scope of a punction. For the remainder, cannulas that are already pre-assembled are easier to keep sterile, because they can be used on the patient more or less directly.

In an advantageous embodiment, the sleeve holder has a holding shoulder, whereby the holding shoulder is provided with a holding block on the side facing away from the sleeve seat. The holding block serves for coupling on a guidance system as described above. Therefore, the sleeve, with the cannula accommodated in it, can be releasably connected with a guidance system that is moved mechanically or by means of an electric motor, by way of the sleeve holder.

The sleeve used in connection with the sleeve holder mentioned above is usually provided with a radially widened sleeve flange at its proximal end, whereby the sleeve flange sits flat on the sleeve seat when assembled as intended, so that the sleeve is maintained in the axial direction, particularly during advance of the cannula through the sleeve. Therefore, when the treating physician introduces the cannula into the patient's body through the sleeve, the sleeve seat of the sleeve holder simultaneously represents a stop for the sleeve accommodated in the sleeve holder.

If the sleeve is moved in the proximal direction after a punction into the patient's skin has already taken place, and thus the sleeve is brought out of engagement with the sleeve seat, the cannula can subsequently be threaded out through the longitudinal slit of the passage, without problems, as already mentioned.

The sleeve seat can also be provided with a triangular recess, whereby the tip of the triangle, which faces the holding shoulder, is preferably provided with a notch crosswise to the sleeve seat. This triangle tip and/or its notch serves for the direct of a cannula or a medical instrument that can be additionally held with a bracket counter-piece pressed down by the treating physician, if necessary. The punction and the further advance can again be carried out manually.

In an alternative embodiment, the sleeve seat can also be provided with an articulated arrangement, in such a manner that the sleeve seat, which is otherwise configured in two-part manner, can be opened and the sleeve can be connected with the sleeve seat. When the articulated arrangement is closed again, a closed passage with the sleeve seat exists. Uncoupling of the guidance system or simply only release of the cannula or of the medical instrument can therefore be implemented, in practical manner, in that the articulated arrangement is opened if necessary.

In another alternative embodiment, the sleeve holder can also be configured as such in multiple parts, in such a manner that the sleeve seat can be uncoupled from the holding shoulder as a whole.

Uncoupling of the sleeve seat from the holding shoulder again takes place simply by means of an axial movement of the sleeve seat relative to the holding shoulder.

In yet another advantageous further development, the holder device can be configured as a controllable gripper element. In this connection, the gripper element can be configured analogous to the mechanism of an iris shutter, such as that used in the sector of single lens reflex cameras.

This gripper element can then in turn be provided with operating elements, such as a voice control or a foot control, and if necessary an optical sensor mechanism. In the latter stage of expansion, it is possible that the system automatically grasps the sleeve handed to it, and performs the interpunction predetermined by the physician, in the orientation predetermined by the physician using an imaging system, at first, and subsequently the physician brings about the advance of the medical instrument or of the cannula, by hand, at this location. In this connection, the gripper element can be opened by way of a corresponding control, at any time, and therefore the medical instrument or the sleeve with the cannula passed through it, respectively, can be uncoupled from the guidance system, for the remainder.

The invention will be explained in greater detail in the following, using an exemplary embodiment that is shown only schematically in the drawing.

This shows:

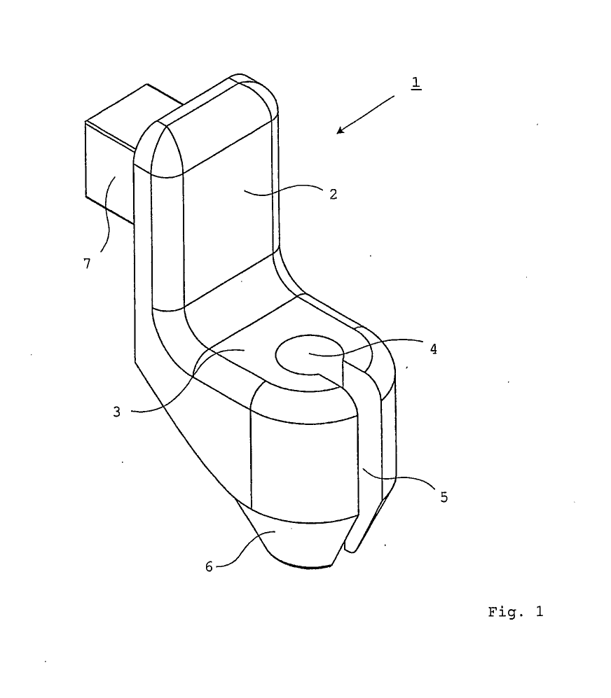

FIG. 1: a holder device for a sleeve having a cannula passed through it, in a perspective representation,

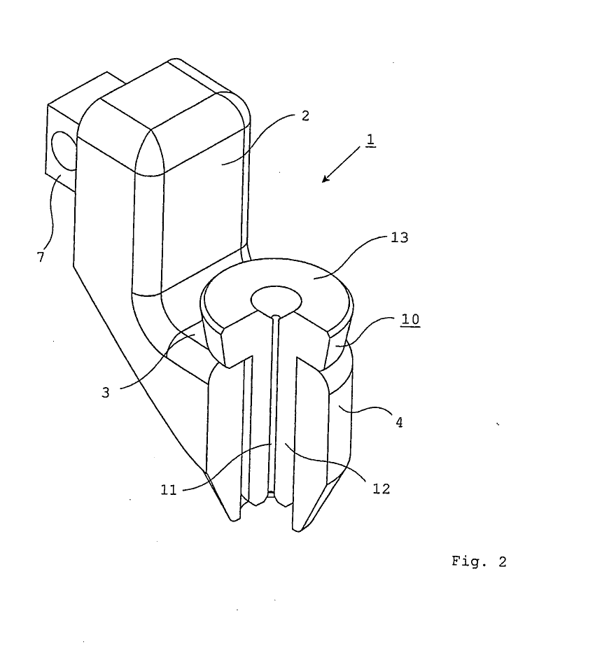

FIG. 2: an alternative embodiment of the holder device with the sleeve held in it, in a perspective representation, with a detail break-down,

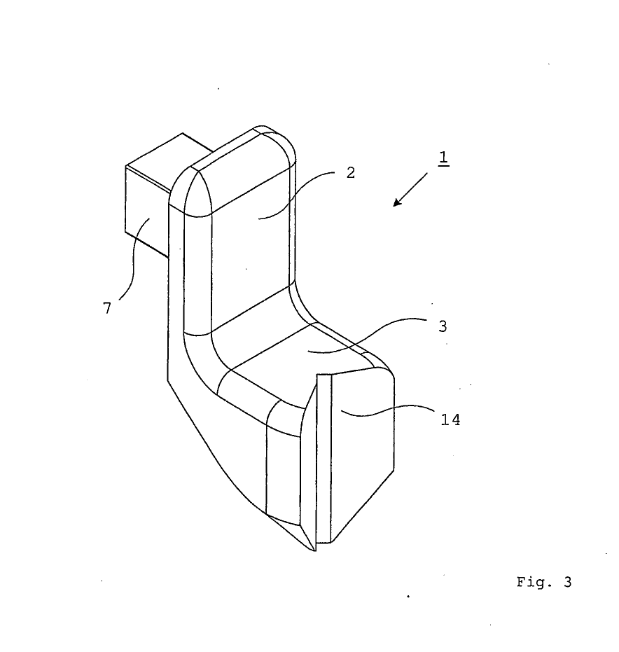

FIG. 3: a holder device having a multi-part sleeve seat, in a perspective representation,

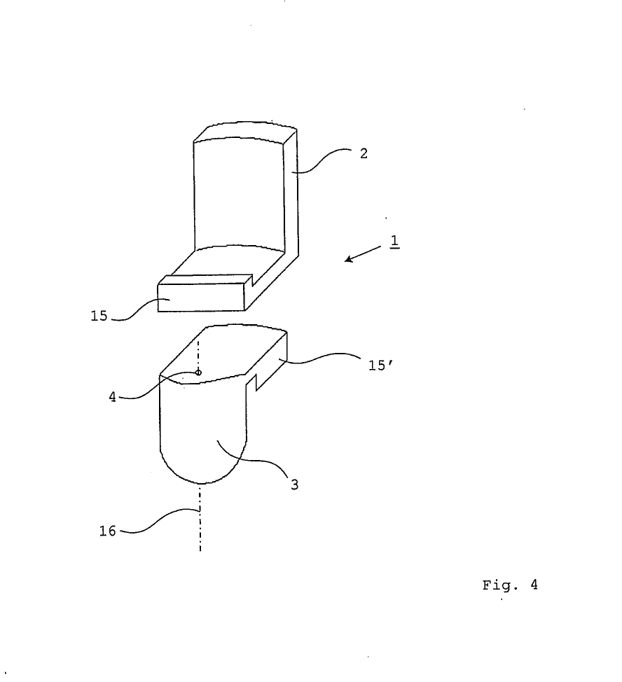

FIG. 4: a holder device having a multi-part sleeve holder, in a perspective representation,

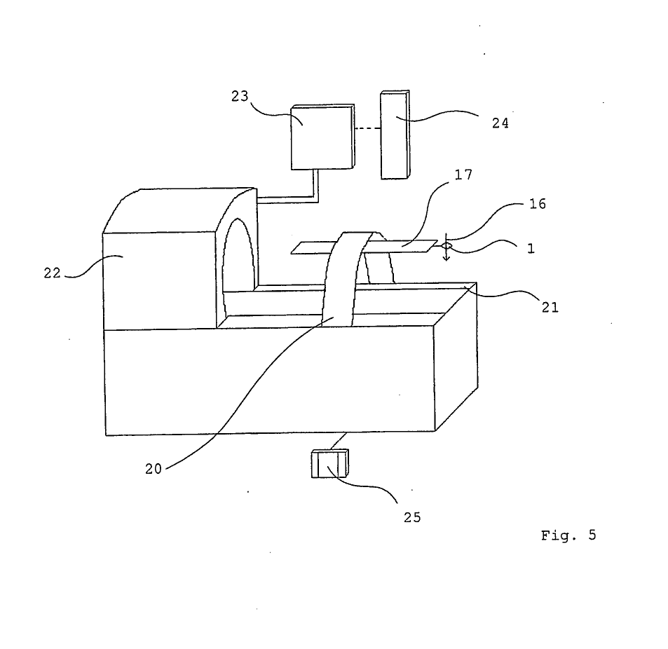

FIG. 5: a fundamental representation of the overall system.

FIG. 1 shows, as an exemplary embodiment of a holder device for medical instruments or cannulas 16, a sleeve holder 1 that is primarily configured as an angular element. The angular element essentially consists of a holding shoulder 2 from which a sleeve seat 3 extends away at an at least approximately right angle. The sleeve seat 3 is completely penetrated more or less centrally by a passage 4, whereby the passage 4 is opened towards the outside by way of a continuous longitudinal slit 5.

On the distal side, which would be the underside of the sleeve seat according to the representation of the figure, a cone 6 is formed onto the sleeve seat 3. This ensures that the cannula can be inserted into the patient's skin even at a comparatively acute angle. The sleeve holder 1 can be connected with a guidance system (17) not shown in detail in FIG. 1. For this purpose, the holding shoulder 2 has a holding block 7 on the side facing away from the sleeve seat 3, for a connection with the guidance system 17.

The sleeve holder (1) shown in FIG. 1 is usually produced from a robust and easy-to-clean plastic, for example by means of a cutting method or as an injection-molded part.

A slightly different embodiment of the sleeve holder 1 is shown in FIG. 2. According to the representation in FIG. 2, a commercially available sleeve 10 is accommodated in the passage 4. The sleeve 10 first of all has a passage hole 11 for accommodation of a segment of a cannula 16, which is not shown in detail in FIG. 2. With regard to the sleeve 10 itself, it is possible to differentiate between a sleeve cylinder 12 that is completely accommodated in the passage 4 of the sleeve holder 1, according to the representation in FIG. 2, and a sleeve flange 13 that is radially widened relative to the sleeve cylinder 12. When the sleeve 10 is assembled as intended, the sleeve flange 13 sits flat on the sleeve seat 3 in the sleeve holder 1. The sleeve seat 3 therefore represents a stop for the sleeve 10, which is particularly useful if a cannula 16 accommodated in the sleeve 10 is advanced in the distal direction by means of the sleeve 10. Usually, the sleeve 10 with the cannula 16 already pre-assembled in it is delivered as a sterile disposable product for one-time use.

Within the scope of the solution according to the invention, such a sleeve 10, with a cannula 16 pre-assembled in it, can be inserted into a sleeve holder 1 according to FIGS. 1 and 2. In this connection, the sleeve holder 1 is usually connected with a guidance system 17, which will be explained below, preferably in releasable manner.

The guidance system is particularly utilized in order to guide the cannula 16 contained in the sleeve holder 1 to a desired interpunction location of the patient and preferably to perform the interpunction itself at that location. Afterwards, the cannula 16 or a medical instrument is advanced into the patient's body up to a desired introduction depth, through the sleeve 10, also by the treating physician. After the intervention has taken place, or if complications occur, or at any time the physician wishes, the sleeve 10 can be released from the sleeve holder 1 in that the sleeve 10 inserted into the passage 4 of the sleeve seat 3 is pulled out upward in the representation of the figure, in other words towards the proximal side, until only the cannula 16, which projects beyond the sleeve 10 on the distal side, is accommodated in the passage 4 of the sleeve seat 3. The cannula 16 can then easily be unthreaded out of the sleeve holder 1, through the longitudinal slit 5 assigned to the passages 4, as intended. In this way, the sleeve 10 with the cannula 16 passed through it has come out of engagement with the sleeve holder 1 and is therefore completely mechanically uncoupled from a guidance system 17 that might be connected with the sleeve holder 1.

In an alternative embodiment according to FIG. 3, the sleeve seat 3 can also be configured in multiple parts. As indicated by the triangular cut-out 14 in the sleeve seat 3, a second sleeve seat part can be connected with the sleeve seat part shown in FIG. 3, at the location of the triangular cut-out 14, by way of an articulated arrangement, not shown here, in such a manner that the cannula 16 itself is accommodated by the passage surrounded by the sleeve seat parts here, in other words without the sleeve 10, by means of opening and closing the articulated arrangement. Holding of the cannula 16 therefore takes place by means of closing the articulated arrangement, while uncoupling of the cannula 16 from the holder 1 is made possible by opening the articulated arrangement.

Alternatively, a counter-piece can also simply be pressed into the triangular cut-out 14 by the physician, by hand, with the interposition of the cannula 16. In order to facilitate this, a notch can additionally be worked into the sleeve seat part 3 shown in FIG. 3, in the axial direction of the cannula 16.

Yet another different configuration of the sleeve holder 1 is shown in FIG. 4. According to the representation in FIG. 4, it is not the sleeve seat 3 but rather the sleeve holder 1 as such that is configured in two parts, as a whole. The angular element of the sleeve holder 1 is only produced in that the holding shoulder 2 is connected with the sleeve seat 3, whereby in this connection, the connection projections 15, 15′ engage into one another, as intended. The sleeve seat 3 can be uncoupled from the holding shoulder 2, as intended, by means of an axial relative movement of the sleeve seat 3 relative to the holding shoulder 2, which is fixed in place, if necessary by means of a connection with a guide system. Such uncoupling of the sleeve seat 3 from the holding shoulder 2 can also take place, for example, by means of a sneeze or careless movement of the patient. However, this is absolutely desirable, since the patient is not supposed to be impaired in any way by means of the positioning aid or the guidance system 17 for introduction of cannulas 16 or medical instruments, particularly not supposed to be fixed in place.

If necessary, a renewed interpunction of the patient might be required after the uncoupling of the sleeve seat 3 from the sleeve holder 1 as described above. Possibly, however, the repositioning of the sleeve holder can also be eliminated, and it might be sufficient to hang the sleeve seat 3 back into the holding shoulder 2.

However, this is intentionally accepted in order to avoid any other impairments of the patient. The dot-dash through the passage 4 of the sleeve seat 3 indicates the position of the cannula 16 in the sleeve seat 3, in sketch-like manner. The embodiment according to FIG. 4 is particularly used for interventions involving respiratory organs.

For the invention to be understood correctly, the sleeve holder 1 must be understood as a component of a comprehensive overall arrangement that is shown in a fundamental diagram in FIG. 5.

The sleeve holder 1 with an instrument or a cannula 16 accommodated in it is mechanically connected with a guidance system 17, in a manner that is of no further interest here. The guidance system 17 permits a translational and rotational movement and is mounted above a treatment table 21 for a patient for this purpose, in correspondingly complicated manner, in a kind of C arch 20. The treatment table 21 additionally has an imaging system, for example a magnetic resonance tomograph 22, assigned to it. The imaging system, in turn, stands in a data connection with display and operating elements, such as a touch screen 23 and/or a computer having a graphic user interface. Both the touch screen 23 and the magnetic resonance tomograph 22 have a data processing system 24 assigned to them. The guidance system 17 can be operated, shut off or turned on by the treating physician, by means of additional operating elements, for example a foot switch 25.

In the following, the function of the overall system will be explained in greater detail, with the appropriate brevity.

Within the framework of a biopsy, which is explained only as an example, the treating physician establishes the location of the interpunction on the patient's body and of the intervention in the patient's body, using an imaging system, for example a magnetic resonance tomograph 22, and marks it on the image representation of the touch screen 23. Using these data, the system then calculates the orientation of the medical instrument to be introduced, or of the cannula 16, into the patient's body, using the data processing system 24, and controls the guidance system 17 accordingly, in such a manner that automatically moves to the patient's body at the desired location, in the required orientation. The interpunction itself, however, is then performed by the physician, as intended. After the interpunction, the physician can then introduce the medical instrument, for example an endoscope, or the cannula 16 described above, supported by the sleeve holder 1 or another holder device 1 intended for the purpose, up to the desired insertion depth. In this connection, because of the special configuration of the holder device 1 according to the invention, the medical instrument or the cannula 16 can be uncoupled from the guidance system 17 or from the holder device 1, respectively, at any time.

### REFERENCE SYMBOL LIST

1 sleeve holder
2 holding shoulder
3 sleeve seat
4 passage
5 longitudinal slit
6 cone
7 holding block
10 sleeve
11 passage bore
12 sleeve cylinder
13 sleeve flange
14 triangular cut-out
15, 15′ holding projection
16 cannula
17 guidance system
20 C arch
21 treatment table
22 magnetic resonance tomograph
23 touch screen
24 data processing device
25 foot switch

1-18. (canceled)

19. Device for holding and/or guiding medical instruments or cannulas (16), which are introduced into a patient's body with a defined orientation, by way of punction, and subsequently driven forward up to a defined penetration depth, within the patient's body, and held in a sleeve holder (1) by means of a sleeve (10),
characterized in that the device is releasable from the instrument or the cannula (16), in accordance with its intended use, in that the sleeve (10) can be displaced relative to the sleeve holder (1), in the direction of the longitudinal axis of the medical instrument or the cannula (16) accommodated in the sleeve, in the proximal direction, to such an extent that the sleeve holder (1) releases the sleeve (10) and the medical instrument or the cannula (16) accommodated in the sleeve (10) can be threaded out through a longitudinal slit (5) of the sleeve holder (1).

20. Device according to claim 19, characterized in that the holder device is in turn connected with a guidance system (17), preferably in releasable manner.

21. Device according to claim 20, characterized in that this guidance system (17) is operated in connection with an imaging system, preferably an MRI (magnetic resonance imaging) system or a CT (computer tomography) system.

22. Device according to claim 21, characterized in that the imaging system has a display and an operating element, preferably a touch screen (23) or a PC having a graphical user interface assigned to it, with the aid of which the punction site and the intervention site on or in the patient's body can be established, whereby the system automatically establishes the positioning and the orientation of the instrument or the cannula at the punction site, on the basis of these data, by means of a computer unit.

23. Device according to claim 22, characterized in that the guidance system (17) automatically moves to the punction site of the patient's body, after the positioning and orientation of the medical instrument or of the cannula (16) have been determined by means of the guidance system (17), in the calculated orientation of the instrument or of the cannula (16), whereby the punction of the patient's body as such preferably remains reserved to the treating physician, in each instance.

24. Device according to claim 23, characterized in that after the punction has taken place, the instrument or the cannula (16) can be introduced into the patient's body to the desired insertion depth, in the predetermined positioning and maintaining the orientation that has been determined, preferably by hand, preferably using the imaging system.

25. Device according to claim 19, characterized in that the device comprises a sleeve holder (1) that essentially is configured as an angular element comprising a holding shoulder (2) and a sleeve seat (3) that extends away from the holding shoulder (2), having a passage (4) for accommodation of at least a segment of a sleeve (10), preferably provided with a cannula (16) that has already been passed through the sleeve (10) in the factory, whereby the passage (4) is provided with the longitudinal slit (5) that extends over the entire longitudinal expanse of this passage (4).

26. Device according to claim 25, characterized in that the holding shoulder (2) of the angular element of the sleeve holder (1) is provided, on the side facing away from the sleeve seat (3), with a holding block (7) that projects on the back side, preferably for a releasable connection with the guidance system (17).

27. Device according to claim 25, characterized in that in the case of assembly as intended, the sleeve (10), which is provided with a radially widened sleeve flange (13) at its proximal end, is held in the sleeve holder (1) in such a manner that the aforementioned sleeve flange (13) sits on the sleeve seat (3) of the sleeve holder (1), preferably in flat manner, while the sleeve (10) for the remainder is accommodated in the passage (4) of the sleeve holder (1), at least in certain segments.

28. Device according to claim 19, characterized in that the sleeve seat (3) is provided with a triangular cut-out (4), whereby the triangle tip 30 that faces the holding shoulder (2) forms the contact surface for a cannula or a medical instrument that can be used by the treating physician, after prior positioning of the sleeve seat, as a positioning and guidance aid, in order to introduce the medical instrument or the cannula, in each instance—with or sleeve—into the patient's body.

29. Device according to claim 28, characterized in that the triangular cut-out can be closed by means of a triangular counter-piece at least essentially corresponding to the triangular cut-out (4), with the interposition of a medical instrument or a cannula (16) held in this manner, preferably by hand.

30. Device according to claim 19, characterized in that the sleeve seat (3) is in two parts, whereby the sleeve seat parts are connected by means of an articulated arrangement, for example by means of a film hinge, in such a manner that a passage (4) for accommodating the sleeve (10) or the cannula (16) as such, in the sleeve seat (3), can be closed and released as necessary.

31. Device according to claim 19, characterized in that the sleeve holder (1) as such is configured in multiple parts, in such a manner that the sleeve seat (3) can be separated from the holding shoulder (2).

32. Device according to claim 31, characterized in that sleeve seat (3) and holding shoulder (2), in each instance, are provided with connection projections that engage into one another, in such a manner that the sleeve seat (3) can be released from the holding shoulder (2) by means of a relative movement in the proximal axial direction of the cannula (16) accommodated in the sleeve seat (3) with introduced cannula (16), and coupled in again by means of a movement in the reverse axial direction.

33. Device according to claim 19, characterized in that the holder (1) is configured for releasable accommodation of the sleeve (10) with cannula (16) passed through it or of a medical instrument, as a mechanically moved gripper element.

34. Device according to claim 33, characterized in that the gripper device is configured in the manner of an iris shutter with a passage (4) that narrows as necessary.

35. Device according to claim 33, characterized in that the gripper device is driven by an electric motor and preferably can be operated by means of a foot control or a voice control, if necessary with the interposition of an optical sensor system.
